SEQUENCE LISTING (47 sequences)

SEQ ID NO: 1 - protein, 155 aa, Artificial Sequence
  Synthetic Peptide
MAEGEITTFTALTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 2 - protein, 141 aa, Artificial Sequence
  Synthetic Peptide
MFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 3 - protein, 239 aa, Homo sapiens
MGLIWLLLLSLLEPGWPAAGPGARLRRDAGGRGGVYEHLGGAPRRRKLYCATKYHLQLHPSGRVNGSLENSAYSILEITAVEVGIVAIRGLFSGRYLAMNKRGRLYASEHYSAECEFVERIHELGYNTYASRLYRTVSSTPGARRQPSAERLWYVSVNGKGRPRRGFKTRRTQKSSLFLPRVLDHRDHEMVRQLQSGLPRPPGKGVQPRRRRQKQSPDNLEPSHVQASRLGSQLEASAH

SEQ ID NO: 4 - protein, 206 aa, Homo sapiens
MSGPGTAAVALLPAVLLALLAPWAGRGGAAAPTAPNGTLEAELERRWESLVALSLARLPVAAQPKEAAVQSGAGDYLLGIKRLRRLYCNVGIGFHLQALPDGRIGGAHADTRDSLLELSPVERGVVSIFGVASRFFVAMSSKGKLYGSPFFTDECTFKEILLPNNYNAYESYKYPGMFIALSKNGKTKKGNRVSPTMKVTHFLPRL

SEQ ID NO: 5 - protein, 268 aa, Homo sapiens
MSLSFLLLLFFSHLILSAWAHGEKRLAPKGQPGPAATDRNPRGSSSRQSSSSAMSSSSASSSPAASLGSQGSGLEQSSFQWSPSGRRTGSLYCRVGIGFHLQIYPDGKVNGSHEANMLSVLEIFAVSQGIVGIRGVFSNKFLAMSKKGKLHASAKFTDDCKFRERFQENSYNTYASAIHRTEKTGREWYVALNKRGKAKRGCSPRVKPQHISTHFLPRFKQSEQPELSFTVTVPEKKKPPSPIKPKIPLSAPRKNTNSVKYRLKFRFG

SEQ ID NO: 6 - protein, 208 aa, Homo sapiens
MALGQKLFITMSRGAGRLQGTLWALVFLGILVGMVVPSPAGTRANNTLLDSRGWGTLLSRSRAGLAGEIAGVNWESGYLVGIKRQRRLYCNVGIGFHLQVLPDGRISGTHEENPYSLLEISTVERGVVSLFGVRSALFVAMNSKGRLYATPSFQEECKFRETLLPNNYNAYESDLYQGTYIALSKYGRVKRGSKVSPIMTVTHFLPRI

SEQ ID NO: 7 - protein, 194 aa, Homo sapiens
MHKWILTWILPTLLYRSCFHIICLVGTISLACNDMTPEQMATNVNCSSPERHTRSYDYMEGGDIRVRRLFCRTQWYLRIDKRGKVKGTQEMKNNYNIMEIRTVAVGIVAIKGVESEFYLAMNKEGKLYAKKECNEDCNFKELILENHYNTYASAKWTHNGGEMFVALNQKGIPVRGKKTKKEQKTAHFLPMAIT

SEQ ID NO: 8 - protein, 244 aa, Homo sapiens
MGSPRSALSCLLLHLLVLCLQAQEGPGRGPALGRELASLFRAGREPQGVSQQVTVQSSPNFTQHVREQSLVTDQLSRRLIRTYQLYSRTSGKHVQVLANKRINAMAEDGDPFAKLIVETDTFGSRVRVRGAETGLYICMNKKGKLIAKSNGKGKDCVFTEIVLENNYTALQNAKYEGWYMAFTRKGRPRKGSKTRQHQREVHFMKRLPRGHHTTEQSLRFEFLNYPPFTRSLRGSQRTWAPEPR

SEQ ID NO: 9 - protein, 208 aa, Homo sapiens
MAPLGEVGNYFGVQDAVPFGNVPVLPVDSPVLLSDHLGQSEAGGLPRGPAVTDLDHLKGILRRRQLYCRTGFHLEIFPNGTIQGTRKDHSRFGILEFISIAVGLVSIRGVDSGLYLGMNEKGELYGSEKLTQECVFREQFEENWYNTYSSNLYKHVDTGRRYYVALNKDGTPREGTRTKRHQKFTHFLPRPVDPDKVPELYKDILSQS

SEQ ID NO: 10 - protein, 208 aa, Homo sapiens
MWKWILTHCASAFPHLPGCCCCCFLLLFLVSSVPVTCQALGQDMVSPEATNSSSSSFSSPSSAGRHVRSYNHLQGDVRWRKLFSFTKYFLKIEKNGKVSGTKKENCPYSILEITSVEIGVVAVKAINSNYYLAMNKKGKLYGSKEFNNDCKLKERIEENGYNTYASFNWQHNGRQMYVALNGKGAPRRGQKTRRKNTSAHFLPMVVHS

SEQ ID NO: 11 - protein, 225 aa, Homo sapiens
MAALASSLIRQKREVREPGGSRPVSAQRRVCPRGTKSLCQKQLLILLSKVRLCGGRPARPDRGPEPQLKGIVTKLFCRQGFYLQANPDGSIQGTPEDTSSFTHFNLIPVGLRVVTIQSAKLGHYMAMNAEGLLYSSPHFTAECRFKECVFENYYVLYASALYRQRRSGRAWYLGLDKEGQVMKGNRVKKTKAAAHFLPKLLEVAMYQEPSLHSVPEASPSSPPAP

SEQ ID NO: 12 - protein, 243 aa, Homo sapiens
MAAAIASSLIRQKRQARESNSDRVSASKRRSSPSKDGRSLCERHVLGVFSKVRFCSGRKRPVRRRPEPQLKGIVTRLFSQQGYFLQMHPDGTIDGTKDENSDYTLFNLIPVGLRVVAIQGVKASLYVAMNGEGYLYSSDVFTPECKFKESVFENYYVIYSSTLYRQQESGRAWFLGLNKEGQIMKGNRVKKTKPSSHFVPKPIEVCMYREPSLHEIGEKQGRSRKSSGTPTMNGGKVVNQDST

SEQ ID NO: 13 - protein, 245 aa, Homo sapiens
MAAAIASSLIRQKRQAREREKSNACKCVSSPSKGKTSCDKNKLNVFSRVKLFGSKKRRRRRPEPQLKGIVTKLYSRQGYHLQLQADGTIDGTKDEDSTYTLFNLIPVGLRVVAIQGVQTKLYLAMNSEGYLYTSELFTPECKFKESVFENYYVTYSSMIYRQQQSGRGWYLGLNKEGEIMKGNHVKKNKPAAHFLPKPLKVAMYKEPSLHDLTEFSRSGSGTPTKSRSVSGVLNGGKSMSHNEST

SEQ ID NO: 14 - protein, 252 aa, Homo sapiens
MVKPVPLFRRTDFKLLLCNHKDLFFLRVSKLLDCFSPKSMWFLWNIFSKGTHMLQCLCGKSLKKNKNPTDPQLKGIVTRLYCRQGYYLQMHPDGALDGTKDDSTNSTLFNLIPVGLRVVAIQGVKTGLYIAMNGEGYLYPSELFTPECKFKESVFENYYVIYSSMLYRQQESGRAWFLGLNKEGQAMKGNRVKKTKPAAHFLPKPLEVAMYREPSLHDVGETVPKPGVTPSKSTSASAIMNGGKPVNKSKTT

SEQ ID NO: 15 - protein, 207 aa, Homo sapiens
MAEVGGVFASLDWDLHGFSSSLGNVPLADSPGFLNERLGQIEGKLQRGSPTDFAHLKGILRRRQLYCRTGFHLEIFPNGTVHGTRHDHSRFGILEFISLAVGLISIRGVDSGLYLGMNERGELYGSKKLTRECVFREQFEENWYNTYASTLYKHSDSERQYYVALNKDGSPREGYRTKRHQKFTHFLPRPVDPSKLPSMSRDLFHYR

SEQ ID NO: 16 - protein, 216 aa, Homo sapiens
MGAARLLPNLTLCLQLLILCCQTQGENHPSPNFNQYVRDQGAMTDQLSRRQIREYQLYSRTSGKHVQVTGRRISATAEDGNKFAKLIVETDTFGSRVRIKGAESEKYICMNKRGKLIGKPSGKSKDCVFTEIVLENNYTAFQNARHEGWFMAFTRQGRPRQASRSRQNQREAHFIKRLYQGQLPFPNHAEKQKQFEFVGSAPTRRTKRTRRPQPLT

SEQ ID NO: 17 - protein, 207 aa, Homo sapiens
MYSAPSACTCLCLHFLLLCFQVQVLVAEENVDFRIHVENQTRARDDVSRKQLRLYQLYSRTSGKHIQVLGRRISARGEDGDKYAQLLVETDTFGSQVRIKGKETEFYLCMNRKGKLVGKPDGTSKECVFIEKVLENNYTALMSAKYSGWYVGFTKKGRPRKGPKTRENQQDVHFMKRYPKGQPELQKPFKYTTVTKRSRRIRPTHPA

SEQ ID NO: 18 - protein, 216 aa, Homo sapiens
MRSGCVVVHVWILAGLWLAVAGRPLAFSDAGPHVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 19 - protein, 211 aa, Homo sapiens
MAPLAEVGGFLGGLEGLGQQVGSHFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 20 - protein, 209 aa, Homo sapiens
MDSDETGFEHSGLWVSVLAGLLLGACQAHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 21 - protein, 170 aa, Homo sapiens
MRRRLWLGLAWLLLARAPDAAGTPSASRGPRSYPHLEGDVRWRRLFSSTHFFLRVDPGGRVQGTRWRHGQDSILEIRSVHVGVVVIKAVSSGFYVAMNRRGRLYGSRLYTVDCRFRERIEENGHNTYASQRWRRRGQPMFLALDRRGGPRPGGRTRRYHLSAHFLPVLVS

SEQ ID NO: 22 - protein, 251 aa, Homo sapiens
MLGARLRLWVCALCSVCSMSVLRAYPNASPLLGSSWGGLIHLYTATARNSYHLQIHKNGHVDGAPHQTIYSALMIRSEDAGFVVITGVMSRRYLCMDFRGNIFGSHYFDPENCRFQHQTLENGYDVYHSPQYHFLVSLGRAKRAFLPGMNPPPYSQFLSRRNEIPLIHFNTPIPRRHTRSAEDDSERDPLNVLKPRARMTPAPASCSQELPSAEDNSPMASDPLGVVRGGRVNTHAGGTGPEGCRPFAKFI

SEQ ID NO: 23 - DNA, 630 bp, Artificial Sequence
  Synthetic Oligonucleotide
gcgtagaggatcgagatctcgatcccgcgaaattaatacgactcactataggggaattgtgagcggataacaattcccctctagaaataattttgtttaactttaagaaggagatatacatatggctgaaggggaaatcaccacctttacagcgttaacggagaaatttaaccttccgcccgggaattacaaaaaacccaagcttctttactgcagtaacggaggacacttcctgcgaattctgccagatggcacagtagatgggactcgcgatcgctccgaccagcacattcagctgcaactctcggccgaaagcgttggagaggtctatatcaagtcgacggagactggccagtaccttgccatggacaccgatgggcttctgtatggctcacagacgcctaacgaagaatgcttgtttctagaaagactagaagaaaaccattacaacacgtacatatcgaaaaaacatgcagagaagaactggtttgtaggccttaaaaaaaatggttcctgtaagcgtggaccacggactcactatggccaaaaggctatcttgttcctgccactaccagtgagctccgactaaggatccgaattcgagctccgtcgacaagcttgcggccgcac

SEQ ID NO: 24 - DNA, 630 bp, Artificial Sequence
  Synthetic Oligonucleotide
gcgtagaggatcgagatctcgatcccgcgaaattaatacgactcactataggggaattgtgagcggataacaattcccctctagaaataattttgtttaactttaagaaggagatatacatatgtttaaccttccgcccgggaattacaaaaaacccaagcttctttactgcagtaacggaggacacttcctgcgaattctgccagatggcacagtagatgggactcgcgatcgctccgaccagcacattcagctgcaactctcggccgaaagcgttggagaggtctatatcaagtcgacggagactggccagtaccttgccatggacaccgatgggcttctgtatggctcacagacgcctaacgaagaatgcttgtttctagaaagactagaagaaaaccattacaacacgtacatatcgaaaaaacatgcagagaagaactggtttgtaggccttaaaaaaaatggttcctgtaagcgtggaccacggactcactatggccaaaaggctatcttgttcctgccactaccagtgagctccgactaaggatccgaattcgagctccgtcgacaagcttgcggccgcactcgagcaccaccaccaccaccactgagatccggctgctaaca

SEQ ID NO: 25 - DNA, 610 bp, Homo sapiens
atgggcctaatctggctgctactgctcagcctgctggagcccggctggcccgcagcgggccctggggcgcggttgcggcgcgatgcgggcggccgtggcggcgtctacgaggggcgccccggcgccgcaagctctactgcgccacgaagtaccacctccaagcggccgcgtcaacggcagcctggagaacagcgcctacagtattttggagtggaggtgggcattgtggccatcaggggtctcttctccgggcggtacctaagaggggacgactctatgcttcggagcactacagcgccgagtgcgagttatccacgagctgggctataatacgtatgcctcccggctgtaccggacggtcctggggcccgccggcagcccagcgccgagagactgtggtacgtgtctgtggccggccccgcaggggcttcaagacccgccgcacacagaagtcctccctcgcgtgctggaccacagggaccacgagatggtgcggcagctacagagtggccccctggtaagggggtccagccccgacggcggcggcagaagcagagcccgagccctctcacgttcaggcttcgagactgggctcccagctggaggccag

SEQ ID NO: 26 - DNA, 619 bp, Homo sapiens
atgtcggggcccgggacggccgcggtagcgctgctcccggcggcctgctggccttgctggcgccctgggcgggccgagggggcgccgccgcacccactgcaccaacggcacgctggaggccgagctggagcgccgctgggagagcctggtggcgctctcgttggcgcgcctgccggtggcagcgcagcccaaggaggcggccgtccagagcggcgccggcgactacctgctgggcatcaagcggctgcggcggctctactgcaacgtgggcatcggcttccacctccaggcgctccccgacggccgcatcggcggcgcgcacgcggacacccgcgacagcctgctggagctctcgcccgtggagcggggcgtggtgagcatcttcggcgtggccagccggttcttcgtggccatgagcagcaagggcaagctctatggctcgcccttcttcaccgatgagtgcacgttcaaggagattctccttcccaacaactacaacgcctacgagtcctacaagtaccccggcatgttcatcgccctgagcaagaatgggaagaccaagaaggggaaccgagtgtcgcccaccatgaaggtcacccacttcctccccaggctgtga

SEQ ID NO: 27 - DNA, 807 bp, Homo sapiens
atgagcttgtccttcctcctcctcctcttcttcagccacctgatcctcagcgcctgggctcacggggagaagcgtctcgcccccaaagggcaacccggacccgctgccactgataggaaccctagaggctccagcagcagacagagcagcagtagcgctatgtcttcctcttctgcctcctcctcccccgcagcttctctgggcagccaaggaagtggcttggagcagagcagtttccagtggagcccctcggggcgccggaccggcagcctctactgcagagtgggcatcggtttccatctgcagatctacccggatggcaaagtcaatggatcccacgaagccaatatgttaagtgttttggaaatatttgctgtgtctcaggggattgtaggaatacgaggagttttcagcaacaaatttttagcgatgtcaaaaaaaggaaaactccatgcaagtgccaagttcacagatgactgcaagttcagggagcgttttcaagaaaatagctataatacctatgcctcagcaatacatagaactgaaaaaacagggcgggagtggtatgtggccctgaataaaagaggaaaagccaaacgagggtgcagcccccgggttaaaccccagcatatctctacccattttctgccaagattcaagcagtcggagcagccagaactttctttcacggttactgttcctgaaaagaaaaagccacctagccctatcaagccaaagattcccctttctgcacctcggaaaaataccaactcagtgaaatacagactcaagtttcgctttggataa

SEQ ID NO: 28 - DNA, 627 bp, Homo sapiens
atggccctgggacagaaactgttcatcactatgtcccggggagcaggacgtctgcagggcacgctgtgggctctcgtcttcctaggcatcctagtgggcatggtggtgccctcgcctgcaggcacccgtgccaacaacacgctgctggactcgaggggctggggcaccctgctgtccaggtctcgcgcggggctagctggagagattgccggggtgaactgggaaagtggctatttggtggggatcaagcggcagcggaggctctactgcaacgtgggcatcggctttcacctccaggtgctccccgacggccggatcagcgggacccacgaggagaacccctacagcctgctggaaatttccactgtggagcgaggcgtggtgagtctctttggagtgagaagtgccctcttcgttgccatgaacagtaaaggaagattgtacgcaacgcccagcttccaagaagaatgcaagttcagagaaaccctcctgcccaacaattacaatgcctacgagtcagacttgtaccaagggacctacattgccctgagcaaatacggacgggtaaagcggggcagcaaggtgtccccgatcatgactgtcactcatttccttcccaggatctaa

SEQ ID NO: 29 - DNA, 585 bp, Homo sapiens
atgcacaaatggatactgacatggatcctgccaactttgctctacagatcatgctttcacattatctgtctagtgggtactatatctttagcttgcaatgacatgactccagagcaaatggctacaaatgtgaactgttccagccctgagcgacacacaagaagttatgattacatggaaggaggggatataagagtgagaagactcttctgtcgaacacagtggtacctgaggatcgataaaagaggcaaagtaaaagggacccaagagatgaagaataattacaatatcatggaaatcaggacagtggcagttggaattgtggcaatcaaaggggtggaaagtgaattctatcttgcaatgaacaaggaaggaaaactctatgcaaagaaagaatgcaatgaagattgtaacttcaaagaactaattctggaaaaccattacaacacatatgcatcagctaaatggacacacaacggaggggaaatgtttgttgccttaaatcaaaaggggattcctgtaagaggaaaaaaaacgaagaaagaacaaaaaacagcccactttcttcctatggcaataacttaa

SEQ ID NO: 30 - DNA, 735 bp, Homo sapiens
atgggcagcccccgctccgcgctgagctgcctgctgttgcacttgctggtcctctgcctccaagcccaggaaggcccgggcaggggccctgcgctgggcagggagctcgcttccctgttccgggctggccgggagccccagggtgtctcccaacaggtaactgttcagtcctcacctaattttacacagcatgtgagggagcagagcctggtgacggatcagctcagccgccgcctcatccggacctaccaactctacagccgcaccagcgggaagcacgtgcaggtcctggccaacaagcgcatcaacgccatggcagaggacggcgaccccttcgcaaagctcatcgtggagacggacacctttggaagcagagttcgagtccgaggagccgagacgggcctctacatctgcatgaacaagaaggggaagctgatcgccaagagcaacggcaaaggcaaggactgcgtcttcacggagattgtgctggagaacaactacacagcgctgcagaatgccaagtacgagggctggtacatggccttcacccgcaagggccggccccgcaagggctccaagacgcggcagcaccagcgtgaggtccacttcatgaagcggctgccccggggccaccacaccaccgagcagagcctgcgcttcgagttcctcaactacccgcccttcacgcgcagcctgcgcggcagccagaggacttgggcccccgagccccgatag

SEQ ID NO: 31 - DNA, 627 bp, Homo sapiens
atggctcccttaggtgaagttgggaactatttcggtgtgcaggatgcggtaccgtttgggaatgtgcccgtgttgccggtggacagcccggttttgttaagtgaccacctgggtcagtccgaagcaggggggctccccaggggacccgcagtcacggacttggatcatttaaaggggattctcaggcggaggcagctatactgcaggactggatttcacttagaaatcttccccaatggtactatccagggaaccaggaaagaccacagccgatttggcattctggaatttatcagtatagcagtgggcctggtcagcattcgaggcgtggacagtggactctacctcgggatgaatgagaagggggagctgtatggatcagaaaaactaacccaagagtgtgtattcagagaacagttcgaagaaaactggtataatacgtactcatcaaacctatataagcacgtggacactggaaggcgatactatgttgcattaaataaagatgggaccccgagagaagggactaggactaaacggcaccagaaattcacacattttttacctagaccagtggaccccgacaaagtacctgaactgtataaggatattctaagccaaagttga

SEQ ID NO: 32 - DNA, 627 bp, Homo sapiens
atgtggaaatggatactgacacattgtgcctcagcctttccccacctgcccggctgctgctgctgctgctttttgttgctgttcttggtgtcttccgtccctgtcacctgccaagcccttggtcaggacatggtgtcaccagaggccaccaactcttcttcctcctccttctcctctccttccagcgcgggaaggcatgtgcggagctacaatcaccttcaaggagatgtccgctggagaaagctattctctttcaccaagtactttctcaagattgagaagaacgggaaggtcagcgggaccaagaaggagaactgcccgtacagcatcctggagataacatcagtagaaatcggagttgttgccgtcaaagccattaacagcaactattacttagccatgaacaagaaggggaaactctatggctcaaaagaatttaacaatgactgtaagctgaaggagaggatagaggaaaatggatacaatacctatgcatcatttaactggcagcataatgggaggcaaatgtatgtggcattgaatggaaaaggagctccaaggagaggacagaaaacacgaaggaaaaacacctctgctcactttcttccaatggtggtacactcatag

SEQ ID NO: 33 - DNA, 678 bp, Homo sapiens
atggcggcgctggccagtagcctgatccggcagaagcgggaggtccgcgagcccgggggcagccggccggtgtcggcgcagcggcgcgtgtgtccccgcggcaccaagtccctttgccagaagcagctcctcatcctgctgtccaaggtgcgactgtgcggggggcggcccgcgcggccggaccgcggcccggagcctcagctcaaaggcatcgtcaccaaactgttctgccgccagggtttctacctccaggcgaatcccgacggaagcatccagggcaccccagaggataccagctccttcacccacttcaacctgatccctgtgggcctccgtgtggtcaccatccagagcgccaagctgggtcactacatggccatgaatgctgagggactgctctacagttcgccgcatttcacagctgagtgtcgctttaaggagtgtgtctttgagaattactacgtcctgtacgcctctgctctctaccgccagcgtcgttctggccgggcctggtacctcggcctggacaaggagggccaggtcatgaagggaaaccgagttaagaagaccaaggcagctgcccactttctgcccaagctcctggaggtggccatgtaccaggagccttctctccacagtgtccccgaggcctccccttccagtccccctgccccctga

SEQ ID NO: 34 - DNA, 732 bp, Homo sapiens
atggctgcggcgatagccagctccttgatccggcagaagcggcaggcgagggagtccaacagcgaccgagtgtcggcctccaagcgccgctccagccccagcaaagacgggcgctccctgtgcgagaggcacgtcctcggggtgttcagcaaagtgcgcttctgcagcggccgcaagaggccggtgaggcggagaccagaaccccagctcaaagggattgtgacaaggttattcagccagcagggatacttcctgcagatgcacccagatggtaccattgatgggaccaaggacgaaaacagcgactacactctcttcaatctaattcccgtgggcctgcgtgtagtggccatccaaggagtgaaggctagcctctatgtggccatgaatggtgaaggctatctctacagttcagatgttttcactccagaatgcaaattcaaggaatctgtgtttgaaaactactatgtgatctattcttccacactgtaccgccagcaagaatcaggccgagcttggtttctgggactcaataaagaaggtcaaattatgaaggggaacagagtgaagaaaaccaagccctcatcacattttgtaccgaaacctattgaagtgtgtatgtacagagaaccatcgctacatgaaattggagaaaaacaagggcgttcaaggaaaagttctggaacaccaaccatgaatggaggcaaagttgtgaatcaagattcaacatag

SEQ ID NO: 35 - DNA, 738 bp, Homo sapiens
atggcggcggctatcgccagctcgctcatccgtcagaagaggcaagcccgcgagcgcgagaaatccaacgcctgcaagtgtgtcagcagccccagcaaaggcaagaccagctgcgacaaaaacaagttaaatgtcttttcccgggtcaaactcttcggctccaagaagaggcgcagaagaagaccagagcctcagcttaagggtatagttaccaagctatacagccgacaaggctaccacttgcagctgcaggcggatggaaccattgatggcaccaaagatgaggacagcacttacactctgtttaacctcatccctgtgggtctgcgagtggtggctatccaaggagttcaaaccaagctgtacttggcaatgaacagtgagggatacttgtacacctcggaacttttcacacctgagtgcaaattcaaagaatcagtgtttgaaaattattatgtgacatattcatcaatgatataccgtcagcagcagtcaggccgagggtggtatctgggtctgaacaaagaaggagagatcatgaaaggcaaccatgtgaagaagaacaagcctgcagctcattttctgcctaaaccactgaaagtggccatgtacaaggagccatcactgcacgatctcacggagttctcccgatctggaagcgggaccccaaccaagagcagaagtgtctctggcgtgctgaacggaggcaaatccatgagccacaatgaatcaacgtag

SEQ ID NO: 36 - DNA, 759 bp, Homo sapiens
atggtaaaaccggtgcccctcttcaggagaactgatttcaaattattattatgcaaccacaaggatctcttctttctcagggtgtctaagctgctggattgcttttcgcccaaatcaatgtggtttctttggaacattttcagcaaaggaacgcatatgctgcagtgtctttgtggcaagagtcttaagaaaaacaagaacccaactgatccccagctcaagggtatagtgaccaggttatattgcaggcaaggctactacttgcaaatgcaccccgatggagctctcgatggaaccaaggatgacagcactaattctacactcttcaacctcataccagtgggactacgtgttgttgccatccagggagtgaaaacagggttgtatatagccatgaatggagaaggttacctctacccatcagaactttttacccctgaatgcaagtttaaagaatctgtttttgaaaattattatgtaatctactcatccatgttgtacagacaacaggaatctggtagagcctggtttttgggattaaataaggaagggcaagctatgaaagggaacagagtaaagaaaaccaaaccagcagctcattttctacccaagccattggaagttgccatgtaccgagaaccatctttgcatgatgttggggaaacggtcccgaagcctggggtgacgccaagtaaaagcacaagtgcgtctgcaataatgaatggaggcaaaccagtcaacaagagtaagacaacatag

SEQ ID NO: 37 - DNA, 624 bp, Homo sapiens
atggcagaggtggggggcgtcttcgcctccttggactgggatctacacggcttctcctcgtctctggggaacgtgcccttagctgactccccaggtttcctgaacgagcgcctgggccaaatcgaggggaagctgcagcgtggctcacccacagacttcgcccacctgaaggggatcctgcggcgccgccagctctactgccgcaccggcttccacctggagatcttccccaacggcacggtgcacgggacccgccacgaccacagccgcttcggaatcctggagtttatcagcctggctgtggggctgatcagcatccggggagtggactctggcctgtacctaggaatgaatgagcgaggagaactctatgggtcgaagaaactcacacgtgaatgtgttttccgggaacagtttgaagaaaactggtacaacacctatgcctcaaccttgtacaaacattcggactcagagagacagtattacgtggccctgaacaaagatggctcaccccgggagggatacaggactaaacgacaccagaaattcactcactttttacccaggcctgtagatccttctaagttgccctccatgtccagagacctctttcactataggtaa

SEQ ID NO: 38 - DNA, 651 bp, Homo sapiens
atgggagccgcccgcctgctgcccaacctcactctgtgcttacagctgctgattctctgctgtcaaactcagggggagaatcacccgtctcctaattttaaccagtacgtgagggaccagggcgccatgaccgaccagctgagcaggcggcagatccgcgagtaccaactctacagcaggaccagtggcaagcacgtgcaggtcaccgggcgtcgcatctccgccaccgccgaggacggcaacaagtttgccaagctcatagtggagacggacacgtttggcagccgggttcgcatcaaaggggctgagagtgagaagtacatctgtatgaacaagaggggcaagctcatcgggaagcccagcgggaagagcaaagactgcgtgttcacggagatcgtgctggagaacaactatacggccttccagaacgcccggcacgagggctggttcatggccttcacgcggcaggggcggccccgccaggcttcccgcagccgccagaaccagcgcgaggcccacttcatcaagcgcctctaccaaggccagctgcccttccccaaccacgccgagaagcagaagcagttcgagtttgtgggctccgcccccacccgccggaccaagcgcacacggcggccccagcccctcacgtag

SEQ ID NO: 39 - DNA, 624 bp, Homo sapiens
atgtattcagcgccctccgcctgcacttgcctgtgtttacacttcctgctgctgtgcttccaggtacaggtgctggttgccgaggagaacgtggacttccgcatccacgtggagaaccagacgcgggctcgggacgatgtgagccgtaagcagctgcggctgtaccagctctacagccggaccagtgggaaacacatccaggtcctgggccgcaggatcagtgcccgcggcgaggatggggacaagtatgcccagctcctagtggagacagacaccttcggtagtcaagtccggatcaagggcaaggagacggaattctacctgtgcatgaaccgcaaaggcaagctcgtggggaagcccgatggcaccagcaaggagtgtgtgttcatcgagaaggttctggagaacaactacacggccctgatgtcggctaagtactccggctggtacgtgggcttcaccaagaaggggcggccgcggaagggccccaagacccgggagaaccagcaggacgtgcatttcatgaagcgctaccccaaggggcagccggagcttcagaagcccttcaagtacacgacggtgaccaagaggtcccgtcggatccggcccacacaccctgcctag

SEQ ID NO: 40 - DNA, 651 bp, Homo sapiens
atgcggagcgggtgtgtggtggtccacgtatggatcctggccggcctctggctggccgtggccgggcgccccctcgccttctcggacgcggggccccacgtgcactacggctggggcgaccccatccgcctgcggcacctgtacacctccggcccccacgggctctccagctgcttcctgcgcatccgtgccgacggcgtcgtggactgcgcgcggggccagagcgcgcacagtttgctggagatcaaggcagtcgctctgcggaccgtggccatcaagggcgtgcacagcgtgcggtacctctgcatgggcgccgacggcaagatgcaggggctgcttcagtactcggaggaagactgtgctttcgaggaggagatccgcccagatggctacaatgtgtaccgatccgagaagcaccgcctcccggtctccctgagcagtgccaaacagcggcagctgtacaagaacagaggctttcttccactctctcatttcctgcccatgctgcccatggtcccagaggagcctgaggacctcaggggccacttggaatctgacatgttctcttcgcccctggagaccgacagcatggacccatttgggcttgtcaccggactggaggccgtgaggagtcccagctttgagaagtaa

SEQ ID NO: 41 - DNA, 636 bp, Homo sapiens
atggctcccttagccgaagtcgggggctttctgggcggcctggagggcttgggccagcaggtgggttcgcatttcctgttgcctcctgccggggagcggccgccgctgctgggcgagcgcaggagcgcggcggagcggagcgcgcgcggcgggccgggggctgcgcagctggcgcacctgcacggcatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacacttga

SEQ ID NO: 42 - DNA, 630 bp, Homo sapiens
atggactcggacgagaccgggttcgagcactcaggactgtgggtttctgtgctggctggtcttctgctgggagcctgccaggcacaccccatccctgactccagtcctctcctgcaattcgggggccaagtccggcagcggtacctctacacagatgatgcccagcagacagaagcccacctggagatcagggaggatgggacggtggggggcgctgctgaccagagccccgaaagtctcctgcagctgaaagccttgaagccgggagttattcaaatcttgggagtcaagacatccaggttcctgtgccagcggccagatggggccctgtatggatcgctccactttgaccctgaggcctgcagcttccgggagctgcttcttgaggacggatacaatgtttaccagtccgaagcccacggcctcccgctgcacctgccagggaacaagtccccacaccgggaccctgcaccccgaggaccagctcgcttcctgccactaccaggcctgccccccgcactcccggagccacccggaatcctggccccccagccccccgatgtgggctcctcggaccctctgagcatggtgggaccttcccagggccgaagccccagctacgcttcctga

SEQ ID NO: 43 - DNA, 513 bp, Homo sapiens
atgcgccgccgcctgtggctgggcctggcctggctgctgctggcgcgggcgccggacgccgcgggaaccccgagcgcgtcgcggggaccgcgcagctacccgcacctggagggcgacgtgcgctggcggcgcctcttctcctccactcacttcttcctgcgcgtggatcccggcggccgcgtgcagggcacccgctggcgccacggccaggacagcatcctggagatccgctctgtacacgtgggcgtcgtggtcatcaaagcagtgtcctcaggcttctacgtggccatgaaccgccggggccgcctctacgggtcgcgactctacaccgtggactgcaggttccgggagcgcatcgaagagaacggccacaacacctacgcctcacagcgctggcgccgccgcggccagcccatgttcctggcgctggacaggaggggggggccccggccaggcggccggacgcggcggtaccacctgtccgcccacttcctgcccgtcctggtctcctga

SEQ ID NO: 44 - DNA, 756 bp, Homo sapiens
atgttgggggcccgcctcaggctctgggtctgtgccttgtgcagcgtctgcagcatgagcgtcctcagagcctatcccaatgcctccccactgctcggctccagctggggtggcctgatccacctgtacacagccacagccaggaacagctaccacctgcagatccacaagaatggccatgtggatggcgcaccccatcagaccatctacagtgccctgatgatcagatcagaggatgctggctttgtggtgattacaggtgtgatgagcagaagatacctctgcatggatttcagaggcaacatttttggatcacactatttcgacccggagaactgcaggttccaacaccagacgctggaaaacgggtacgacgtctaccactctcctcagtatcacttcctggtcagtctgggccgggcgaagagagccttcctgccaggcatgaacccacccccgtactcccagttcctgtcccggaggaacgagatccccctaattcacttcaacacccccataccacggcggcacacccggagcgccgaggacgactcggagcgggaccccctgaacgtgctgaagccccgggcccggatgaccccggccccggcctcctgttcacaggagctcccgagcgccgaggacaacagcccgatggccagtgacccattaggggtggtcaggggcggtcgagtgaacacgcacgctgggggaacgggcccggaaggctgccgccccttcgccaagttcatctag

SEQ ID NO: 45 - protein, 6 aa, Homo sapiens
HHHHHH

SEQ ID NO: 46 - protein, 5 aa, Homo sapiens
DDDDK

SEQ ID NO: 47 - protein, 6 aa, Homo sapiens
ADDDDK

(19) United States
(12) Patent Application Publication
(10) Pub. No.: US 2015/0104494 A1
(43) Pub. Date: Apr. 16, 2015

(54) METHODS FOR REPAIR OF EAR CANAL TISSUE DEFECTS

(71) Applicants: Ohio State Innovation Foundation, Columbus, OH (US); New Technologies Holding, Pte. Ltd., Singapore (SG)

(72) Inventors: James C. Oliver, Raleigh, NC (US); Judi Appleman, Irvine, CA (US); Wei Chen, San Diego, CA (US); D. Bradley Welling, Columbus, OH (US)

(21) Appl. No.: 14/514,309

(22) Filed: Oct. 14, 2014

Related U.S. Application Data
(60) Provisional application No. 61/889,534, filed on Oct. 11, 2013.

(51) Int. Cl. A61K 38/18; A61K 9/00; A61K 9/70

(52) U.S. Cl. CPC A61K 38/1825; A61K 9/70; A61K 9/0046
USPC 424/443; 514/9.1

(57) ABSTRACT

Disclosed herein are methods of repairing ear canal tissue defects by administering non-basic fibroblast growth factor (FGF) to the ear canal tissue defect. Also disclosed are delivery devices to administer said non-basic FGF.

### CROSS REFERENCE TO RELATED APPLICATION

This application claims priority to U.S. Provisional Application No. 61/889,534, filed Oct. 11, 2013, which is incorporated herein by reference in its entirety.

### SEQUENCE LISTING

The sequence listing is filed with the application in electronic format only and is incorporated by reference herein. The sequence listing text file “ASFILED_SequenceListing_Text” was created on Oct. 14, 2014 and is 63,931 bytes in size.

### TECHNICAL FIELD

The present invention relates to methods of non-surgical repair of ear canal tissue defects comprising administering a non-basic fibroblast growth factor.

### BACKGROUND

Tympanic membrane perforation (TMP) is the most common primary blast injury in the current conflicts in Afghanistan and Iraq occurring in 10-35% of service members wounded by combat explosions. The tympanic membrane, also called the eardrum, is a flexible, translucent, diaphragm like structure. TMPs can result from disease, trauma, or medical care. Perforations can be temporary or persistent. Effect varies with size, location on the drum surface, and associated pathologic condition. Perforation symptoms include audible whistling sounds during sneezing and nose blowing, decreased hearing, especially with larger perforations and a tendency to become infected during colds and when water enters the ear canal. Hearing loss may be present, especially with larger perforations.

Current methods for repairing substantial ear canal tissue defects, such as chronic perforated tympanic membranes, require surgical intervention. However, since these require general anesthesia and are performed in a surgical center, they are costly and recovery is painful. Thus, there is a need in the art for non-surgical methods for repair of such ear canal tissue defects.

### SUMMARY

The present invention is directed to a method for treating an ear canal tissue defect in a subject. The method comprises administering to the subject an amount effective of non-basic fibroblast growth factor (FGF) to treat the ear canal tissue defect. The non-basic FGF may comprise FGF-1 (SEQ ID NO: 1) or a fragment thereof. The non-basic FGF may comprise a polypeptide sequence of amino acids 15-155 of SEQ ID NO: 1. The non-basic FGF may consist of a polypeptide sequence of amino acids 15-155 of SEQ ID NO: 1. The ear canal tissue defect may comprise a perforated tympanic membrane. The non-basic FGF may be administered to the perforated tympanic membrane. The non-basic FGF may be administered using a delivery device. The delivery device may be a gelatin sponge. The delivery device may further comprise heparin. The delivery device may comprise a covering material. The covering material may comprise a fibrin glue. The effective amount of non-basic FGF may comprise between about 25 μg/mL and 100 μg/mL. The method may further comprise creating a fresh wound at the site of defect before administering the non-basic FGF.

The present invention is directed to a gelatin sponge coated or impregnated with a non-basic FGF. The non-basic FGF may comprise FGF-1 (SEQ ID NO: 1) or a fragment thereof. The non-basic FGF may consist of a polypeptide sequence of amino acids 15-155 of SEQ ID NO: 1. The gelatin sponge may be impregnated with between about 25 g/mL and about 100 μg/mL non-basic FGF. The gelatin sponge may further comprise heparin. The gelatin sponge may comprise a covering material. The covering material may comprise a fibrin glue.

### DETAILED DESCRIPTION

The present disclosure provides a non-surgical method for repair of ear canal tissue defects. The method involves a procedure of administering a non-basic Fibroblast growth factor (FGF), such as FGF-1, to the subject with the ear canal tissue defect. The non-basic FGF may be applied to or over the perforated membrane using a delivery system, such as a gel foam or gelatin sponge moistened with the non-basic FGF. This in-office procedure is simpler, not as costly, and less painful to the subject, and is associated with a lower risk of complications.

### 1. DEFINITIONS

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Covering material” as used herein refers to a material that covers the gelatin sponge to prevent drying and infections and to create a good culturing environment for the tympanic membrane regeneration, which is isolated from the outside.

As used herein, an “ear canal tissue defect” comprises any significant tissue defect to an ear canal tissue, including but not limited to perforated tympanic membrane, and ear canal bone exposure after ear canal cholesteatoma resection, tympanoplasty or ear canal tumor resection.

“Effective dosage” as used herein means a dosage of a drug effective for periods of time necessary, to achieve the desired therapeutic result. An effective dosage may be determined by a person skilled in the art and may vary according to factors such as the disease state, age, sex, and weight of the individual, and the ability of the drug to elicit a desired response in the individual. This term as used herein may also refer to an amount effective at bringing about a desired in vivo effect in an animal, mammal, or human, such as reducing and/or inhibiting the function of the estrogen receptor. A therapeutically effective amount may be administered in one or more administrations (e.g., the agent may be given as a preventative treatment or therapeutically at any stage of disease progression, before or after symptoms, and the like), applications or dosages and is not intended to be limited to a particular formulation, combination or administration route. It is within the scope of the present disclosure that the non-basic FGF may be administered at various times during the course of treatment of the subject. The times of administration and dosages used will depend on several factors, such as the goal of treatment (e.g., treating v. preventing), condition of the subject, etc. and can be readily determined by one skilled in the art.

“Fibrin glue” (also known as fibrin sealant) as used herein refers to a formulation used to create a fibrin clot. Fibrin glue is made of fibrinogen (such as lyophilized pooled human concentrate) and thrombin (such as bovine, which is reconstituted with calcium chloride) that are applied to the tissue sites to glue them together.

“Fibroblast growth factor” or “FGF” as used interchangeably herein refers to a family of growth factors, with members involved in angiogenesis, wound healing, embryonic development and various endocrine signaling pathways. FGF family members possess broad mitogenic and cell survival activities, and are involved in a variety of biological processes, including embryonic development, cell growth, morphogenesis, tissue repair, tumor growth and invasion. The FGFs are heparin-binding proteins and interactions with cell-surface-associated heparan sulfate proteoglycans have been shown to be essential for FGF signal transduction. FGFs are key players in the processes of proliferation and differentiation of wide variety of cells and tissues. For example, FGF-1 (also known as acidic FGF) functions as a modifier of endothelial cell migration and proliferation, as well as an angiogenic factor. It acts as a mitogen for a variety of mesoderm- and neuroectoderm-derived cells in vitro, thus is thought to be involved in organogenesis.

As used herein, a “gelatin sponge” is any biocompatible gelatin-containing delivery device.

“Heparin” as used herein refers to an anticoagulant that is a highly sulfated glycosaminoglycan. Heparin reduces the blood's ability to clot so that clots cannot form in the veins, arteries or lungs. This drug can be used during surgical procedures that create a high risk of blood clot formation. It can also be used to treat heart, lung and blood vessels conditions that increase the likelihood of dangerous clots.

The terms “homology” or “similarity” as used herein refer to the degree of sequence similarity between two polypeptides or between two nucleic acid molecules compared by sequence alignment. The degree of homology between two discrete nucleic acid sequences being compared is a function of the number of identical, or matching, nucleotides at comparable positions.

“Heterologous” as used herein with respect to a sequence means a sequence that originates from a foreign species, or, if from the same species, is substantially modified from its native form in composition and/or genomic locus by deliberate human intervention.

“Identical” or “identity,” as used herein in the context of two or more polypeptide or polynucleotide sequences, can mean that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of the single sequence are included in the denominator but not the numerator of the calculation.

The terms “isolated,” “purified” or “biologically pure” as used herein refer to material that is substantially or essentially free from components that normally accompany it as found in its native state. Purity and homogeneity are typically determined using analytical chemistry techniques such as polyacrylamide gel electrophoresis or high performance liquid chromatography. A protein that is the predominant species present in a preparation is substantially purified. The term “purified” as used herein denotes that a nucleic acid or protein gives rise to essentially one band in an electrophoretic gel. Particularly, it means that the nucleic acid or protein is at least 85% pure, more preferably at least 95% pure, and most preferably at least 99% pure.

“Linking sequence” or “linking peptide sequence” refers to a natural or artificial polypeptide sequence that is connected to one or more polypeptide sequences of interest (e.g., full-length, fragments, etc.). The term “connected” refers to the joining of the linking sequence to the polypeptide sequence of interest. Such polypeptide sequences are preferably joined by one or more peptide bonds. Linking sequences can have a length of from about 4 to about 50 amino acids. Preferably, the length of the linking sequence is from about 6 to about 30 amino acids. Natural linking sequences can be modified by amino acid substitutions, additions, or deletions to create artificial linking sequences. Exemplary linking sequences include, but are not limited to: (i) Histidine (His) tags, such as a 6× His tag, which has an amino acid sequence of HHHHHH (SEQ ID NO: 45), are useful as linking sequences to facilitate the isolation and purification of polypeptides of interest; (ii) Enterokinase cleavage sites, like His tags, are used in the isolation and purification of proteins of interest. Often, enterokinase cleavage sites are used together with His tags in the isolation and purification of proteins of interest. Various enterokinase cleavage sites are known in the art. Examples of enterokinase cleavage sites include, but are not limited to, the amino acid sequence of DDDDK (SEQ ID NO: 46) and derivatives thereof (e.g., ADDDDK (SEQ ID NO: 47), etc.); (iii) Miscellaneous sequences can be used to link or connect the proteins of interest. Examples of other linking sequences can be found in Bird et al., Science 242: 423-426 (1988); Huston et al., PNAS USA 85: 5879-5883 (1988); and McCafferty et al., Nature 348: 552-554 (1990). Linking sequences also can be modified for additional functions, such as attachment of drugs or attachment to solid supports. In the context of the present disclosure, the non-basic FGF, for example, can contain a linking sequence, such as a His tag, an enterokinase cleavage site, or both.

“Non-basic FGF” refers to a FGF that is not basic FGF or FGF-2.

The term “polypeptide” as used herein refers to a sequence of subunit amino acids. The polypeptides of the invention may comprise L-amino acids, D-amino acids (which are resistant to L-amino acid-specific proteases in vivo), or a combination of D- and L-amino acids. The polypeptides described herein may be obtained from naturally occurring sources, chemically synthesized, or recombinantly expressed.

The term “mutant FGF polypeptide” or “FGF variant” as used interchangeably herein includes a fibroblast growth factor (FGF) from human and includes polypeptides comprising, consisting or consisting essentially of polypeptides with substantial homology (that is, sequence similarity) or substantial identity to one or more of SEQ ID NOs: 1-22, or polypeptide variants that have at least about 60%, 61%, 62%, 63% 64%, 65%, 66%, 67%, 68%, 69%, 70%, 75%, 80%, 85%, 87%, 88%, 89%, 90%, 91%, 92%, 93%, 94%, 95% 96%, 97%, 98% or 99%, or from about 60% to about 99%, from about 70% to about 90%, or from about 80% to about 99% sequence identity to the sequence of one or more of SEQ ID NOs: 1-22; fragments of the polypeptides including fragments of one or more of SEQ ID NOs: 1-22; and fragments of one or more of SEQ ID NOs: 1-22 with substantial homology (that is, sequence similarity) or substantial identity thereto that have at least about 60%, 61%, 62%, 63%, 64%, 65%, 66%, 67%, 68%, 69%, 70%, 75%, 80%, 85%, 87%, 88%, 89%, 90%, 91%, 92%, 93%, 94%, 95% 96%, 97%, 98%, 99% or 100%, or from about 60% to about 100%, from about 70% to about 90%, or from about 80% to about 100% sequence identity to the corresponding fragments of one or more of SEQ ID NOs: 1-22. The fragments may be at least about 10, 20, 30, 40, 50, 60, 70, 80, 90, 100, 110, 120, 130, 135, 140, 145, 150, 155, 160, 165, 170, 175, 180, 190, 200, or 205 amino acid residues in length, e.g. the mutant FGF may comprise (i) at least about 10, 20, 30, 40, 50, 60, 70, 80, 90, 100, 110, 120, 130, 135, 140, 145, 150, 155, 160, 165, 170, 175, 180, 190, 200, or 205 amino acids of one or more of SEQ ID NOs: 1-22, or (ii) a sequence having at least about 60%, 61%, 62%, 63%, 64%, 65%, 66%, 67%, 68%, 69%, 70%, 75%, 80%, 85%, 87%, 88%, 89%, 90%, 91%, 92%, 93%, 94%, 95% 96%, 97%, 98%, 99% or 100%, or from about 60% to about 100%, from about 70% to about 90%, or from about 80% to about 100% sequence identity to at least about 10, 20, 30, 40, 50, 60, 70, 80, 90, 100, 110, 120, 130, 135, 140, 145, 150, 155, 160, 165, 170, 175, 180, 190, 200, or 205 amino acids of one or more of SEQ ID NOs: 1-22. Typically the fragment may retain the biological activity of the full length sequence, e.g. the fragment comprises FGF activity, typically modified FGF activity. The non-basic FGF polypeptide also includes sequences comprising a sufficient or substantial degree of identity or similarity to one or more of SEQ ID NOs: 1-22 that functions as a FGF. In one embodiment, the term “non-basic FGF polypeptide” refers to a polymer of amino acids which comprises, consists or consists essentially of a polypeptide designated herein as one or more of SEQ ID NOs: 1-22.

The term “FGF variant polynucleotide” includes polynucleotides encoding a mutant FGF or FGF variant and includes polynucleotides comprising, consisting or consisting essentially of polynucleotides with substantial homology (that is, sequence similarity) or substantial identity to one or more of SEQ ID NOs: 23-44, or polynucleotide variants that have at least about 60%, 61%, 62%, 63% 64%, 65%, 66%, 67%, 68%, 69%, 70%, 75%, 80%, 85%, 87%, 88%, 89%, 90%, 91%, 92%, 93%, 94%, 95% 96%, 97%, 98% or 99%, or from about 60% to about 99%, from about 70% to about 90%, or from about 80% to about 99% sequence identity to the sequence of one or more of SEQ ID NOs: 23-44, fragments of the polynucleotides including fragments of one or more of SEQ ID NOs: 23-44, and fragments of one or more of SEQ ID NOs: 23-44 with substantial homology (that is, sequence similarity) or substantial identity thereto that have at least about 60%, 61%, 62%, 63%, 64%, 65%, 66%, 67%, 68%, 69%, 70%, 75%, 80%, 85%, 87%, 88%, 89%, 90%, 91%, 92%, 93%, 94%, 95% 96%, 97%, 98%, 99% or 100%, or from about 60% to about 100%, from about 70% to about 90%, or from about 80% to about 100% sequence identity to the corresponding fragments of one or more of SEQ ID NOs: 23-44. The fragments may be at least about 20, 50, 70, 100, 200, 300, 400, 500, 600, 700, or 800 nucleotides in length, e.g. the FGF variant polynucleotide may comprise (i) at least about 20, 50, 70, 100, 200, 300, 400, 500, 600, 700, or 800 nucleotides of one or more of SEQ ID NOs: 23-44, or (ii) a sequence having at least about 60%, 61%, 62%, 63%, 64%, 65%, 66%, 67%, 68%, 69%, 70%, 75%, 80%, 85%, 87%, 88%, 89%, 90%, 91%, 92%, 93%, 94%, 95% 96%, 97%, 98%, 99% or 100%, or from about 60% to about 100%, from about 70% to about 90%, or from about 80% to about 100% sequence identity to at least about 20, 50, 70, 100, 200, 300, 400, 500, 600, 700, or 800 nucleotides of one or more of SEQ ID NOs: 23-44. Typically the fragment may encode a polypeptide that retains the biological activity of the full length polypeptide sequence, e.g. the fragment encodes a polypeptide that has FGF activity. The FGF variant polynucleotide also includes sequences comprising a sufficient or substantial degree of identity or similarity to one or more of SEQ ID NOs: 23-44 to encode a polypeptide that functions as a FGF. In one embodiment, the term “FGF-1 polynucleotide” refers to a polymer of nucleotides which comprises, consists or consists essentially of a polynucleotide designated herein as one or more of SEQ ID NOs: 23 or 24.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc) and a human). In some embodiments, the subject may be a human or a non-human. The subject or patient may be undergoing other forms of treatment.

“Tympanic membrane perforation” or “TMP” as used interchangeably herein refers to a temporary or persistent perforation of the tympanic membrane. TMPs may result from disease, such as infection, trauma, such as blows to the ear, severe atmospheric overpressure, exposure to excessive water pressure, and improper attempts at ear cleaning or wax removal, or medical care. The effect of the TMP may vary with size, location on the drum surface, and associated pathologic condition.

“Treat”, “treating” or “treatment” are each used interchangeably herein to describe reversing, alleviating, or inhibiting the progress of a disease or injury, or one or more symptoms of such disease or injury, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of FGF or pharmaceutical composition thereof to a subject that is not at the time of administration afflicted with the disease. “Preventing” also refers to preventing the recurrence of a disease or of one or more symptoms associated with such disease or injury. “Treatment” and “therapeutically,” refer to the act of treating, as “treating” is defined above.

“Variant” is used herein to describe a peptide or polypeptide that differs in amino acid sequence by the insertion, deletion, or conservative substitution of amino acids, but retain at least one biological activity. Representative examples of “biological activity” include the ability to be bind to FGF receptor, heparin, and/or heparan sulfate proteoglycans. Variant is also used herein to describe a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree, and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. Substitution of amino acids having similar hydrophilicity values can result in peptides retaining biological activity, as is understood in the art. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties. “Variant” also can be used to describe a polypeptide or a fragment thereof that has been differentially processed, such as by proteolysis, phosphorylation, or other post-translational modification, yet retains its biological activity.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

### 2. METHODS FOR TREATING EAR CANAL TISSUE DEFECTS

The present invention provides methods for treating an ear canal tissue defect, comprising administering to a subject with an ear canal tissue defect an effective amount of non-basic fibroblast growth factor (FGF) to treat the ear canal tissue defect. As used herein, “treating” means accomplishing one or more of the following: (a) repairing the ear canal defect; (b) improving tone/speech discrimination; and (c) improving tympanic membrane mobility.

In another embodiment, the methods further comprise creating a fresh wound at the site of defect. For example, in cases where the defect is not recent, it is preferable that the defect margin is “freshened” to promote tissue regeneration, such as by removing/injuring the margin using a surgical knife or similar instrument, or by administering a protein-denaturant, such as aluminum acetate or a high concentration of a local anesthetic.

### 3. EAR CANAL TISSUE DEFECT

In a preferred embodiment, the defect comprises a perforated tympanic membrane, and more preferably a chronic, non-healing defect and/or an acutely perforated tympanic membrane. The tympanic membrane has a 3-layer structure, which is necessary to ensure optimal sound conduction. Cases of spontaneous cure of tympanic membrane perforations without medical intervention often involve only regeneration of the epithelial layer, resulting in reduced sound conduction. The methods of the present invention serve to repair the three-layer structure. Such a perforated tympanic membrane may be the result of any type of injury, including but not limited to include chronic otitis media, re-perforation after tympanic membrane closure surgery or tympanoplasty, old traumatic tympanic membrane perforation, perforation remaining after tympanic membrane incision or tympanic membrane tube indwelling for otitis media with effusion, ear infections, acoustic trauma, barotrauma, foreign objects in ear, other ear injury, and the like.

The methods of the present invention are particularly suitable for subjects with an ear canal soft tissue defect with a diameter of about 5 mm or more, such as a diameter of about 5.0 mm, about 5.1 mm, about 5.2 mm, about 5.3 mm, about 5.4 mm, about 5.5 mm, about 5.6 mm, about 5.7 mm, about 5.8 mm, about 5.9 mm, about 6.0 mm, about 6.5 mm, or about 7 mm, and/or for subjects with a defect affecting 30% or more of the tympanic membrane, such as a defect affecting about 30%, about 35%, about 40%, about 45%, about 50%, about 55%, about 60%, about 65%, about 70%, about 75%, about 80%, about 85%, about 90%, about 95%, or about 99% of the tympanic membrane.

The tympanic membrane/ear canal regeneration agent of the present invention is applicable in all cases, as far as the patient's tympanic membrane perforation or ear canal soft tissue defect is not accompanied by active infections/inflammation in the middle ear/external ear. For example, cases of tympanic membrane perforation include chronic otitis media, re-perforation after tympanic membrane closure surgery or tympanoplasty, old traumatic tympanic membrane perforation, perforation remaining after tympanic membrane incision or tympanic membrane tube indwelling for otitis media with effusion and the like. Example cases of ear canal soft tissue defect include those with an ear canal soft tissue defect and ear canal bone exposure after ear canal cholesteatoma resection, tympanoplasty or ear canal tumor resection.

The tympanic membrane/ear canal regeneration agent of the present invention can preferably be used particularly for patients with a large tympanic membrane perforation or ear canal soft tissue defect for which therapies using a conventional regenerative medical technique are not indicated. Specifically, in patients with a tympanic membrane perforation wherein more than ⅓, particularly more than ⅔, of the tympanic membrane is defective, repair of the tympanic membrane is possible. In patients having an ear canal soft tissue defect having a maximum diameter of 1 cm or more, particularly 2 cm or more, repair of the ear canal is possible.

### 4. FIBROBLAST GROWTH FACTOR

The non-basic FGF for use in the methods of the present invention can be any FGF other than FGF-2 (also known as basic FGF). Exemplary human non-basic FGFs for use in the methods of the invention include FGF-1, FGF-3 through 14, and FGF-16 through 23 (SEQ ID NOs: 1-22). The FGF-1 sequence may be chemically synthesized with alterations for preferred codon usage in E. coli. The other non-basic FGF sequences may be native sequences obtained from NCBI. FGF-1 through FGF-10 bind fibroblast growth factor receptors (FGFRs). FGF11, FGF12, FGF13, and FGF14, also known as FGF homologous factors 1-4 (FHF1-FHF4) and “iFGF”, have been shown to have distinct functional differences compared to the FGFs. Although these factors possess remarkably similar sequence homology, they do not bind FGFRs and are involved in intracellular processes unrelated to the FGFs. Human FGF-18 is involved in cell development and morphogenesis in various tissues including cartilage. Human FGF20 was identified based on its homology to Xenopus FGF-20 (XFGF-20). FGF15 is the mouse ortholog of human FGF19 (there is no human FGF15) and, where their functions are shared, they are often described as FGF15/19. In contrast to the local activity of the other FGFs, FGF15/19, FGF21 and FGF23 have systemic effects.

In a preferred embodiment, the non-basic FGF is human acidic fibroblast growth factor (FGF-1). In one embodiment, the non-basic FGF is a 155 amino acid FGF-1 polypeptide (FGF-1₁₅₅; SEQ ID NO: 1). In some embodiments, the non-basic FGF is a 141 amino acid FGF-1 polypeptide (FGF-1₁₄₁; SEQ ID NO:2), which contains an N-terminal Met residue followed by amino acid residues 15-155 of FGF-1₁₅₅. In another embodiment, FGF-1 is a 140 amino acid FGF-1 polypeptide (FGF-1₁₅₋₁₅₅), that consists of residues 15-155 of FGF-1₁₅₅, and is believed to be naturally produced as a proteolytic product of FGF-1₁₅₅.

In further embodiments, a non-basic FGF homolog or mutant may be used, such as a homolog or mutant of any of the non-basic FGF species described above. Any suitable non-basic FGF homolog or mutant that maintains the biological activity of the non-basic FGF, such as FGF-1, can be used. In one non-limiting embodiment, a polypeptide can include amino acid substitutions, deletions, insertion, etc., that do not result in decreased FGF activity. In some embodiments, the non-basic FGF homolog is a heterologous non-basic FGF. Similarly, the non-basic FGF may be modified with non-amino acid components as suitable for a given purpose. For example, the polypeptides may be linked to other compounds to promote an increased half-life in vivo, such as by PEGylation, HESylation, PASylation, glycosylation, etc. Such linkage can be covalent or non-covalent as is understood by those of skill in the art.

### 5. COMBINATION TREATMENT

The non-basic FGF may be used in combination with another compound or active agents. In some embodiments, the non-basic FGF is used in combination with a compound that provides FGF stability and potency. For example, the non-basic FGF may be used in combination with heparin, which improves FGF stability and potency. The non-basic FGF may be formulated in sterile phosphate buffered saline (PBS) with the addition of heparin at a weight ratio of 3 parts heparin to 1 part FGF, such as FGF-1, including FGF-1₁₄₀ or FGF-1₁₄₁.

### 6. DELIVERY SYSTEMS

In a preferred embodiment, the non-basic FGF, such as FGF-1, is present administered using a delivery device. Any delivery device suitable for delivery to a subject's ear canal can be used, including but not limited to gel foams and other biocompatible scaffold materials (collagen membranes, chitin membranes, cellulostic membranes, etc.). In a preferred embodiment, the delivery device comprises a gelatin sponge.

In another embodiment, the device, such as a gelatin sponge, can be covered (in whole or in part) with a covering material. This helps to prevent/limit drying of the device and helps to limit infections. The covering material can be any suitable biocompatible material that can help to adhere the device to the defect. In one embodiment, the covering material comprises a commercially available fibrin glue (blood fibrinogen and thrombin extracts) or commercially available water soluble polymeric polysaccharide (including but not limited to chitin, chitosan or alginic acid or a salt thereof), combinations thereof, or derivatives thereof. The covering material may be applied to the FGF-containing delivery device via any suitable means. For example, when the covering material is liquid, it can be administered by dripping several drops thereof on the surface of the delivery device indwelled in the defective portion, by spraying it using a sprayer, etc. When the covering material is a sheet-like substance, it can be placed on the surface of the delivery device indwelled in the defective portion is. In another embodiment that can be combined with any of the above embodiments, a covering material is present over the sponge. In a preferred embodiment, the covering material comprises fibrin glue.

### 7. GELATIN SPONGE

In another aspect, the present invention provides a gelatin sponge coated with non-basic FGF, such as FGF-1. All embodiments of the gelatin sponge and FGF-1 described above are equally applicable in this aspect. In this embodiment, the gelatin sponge is used as a malleable scaffold material that permits covering the entire defect (such as a perforated tympanic membrane) to be covered. Such a gelatin sponge is coated or impregnated with non-basic FGF, such as an FGF-1 solution. In a further preferred embodiment, the gelatin sponge is a porous structure, such that it can act as a cell growth scaffold without interfering with cell elongation, permitting repair of large defects for which no other consistently effective non-surgical treatment is presently available. In various embodiments, the mean pore diameter is about 10 μm to about 500 μm; preferably about 100 μm to about 400 μm.

The non-basic FGF-carrying gelatin sponge may be adjusted to a size larger than the defective portion of the tympanic membrane or ear canal, and is indwelled in a way such that the entire defective portion is covered. Here, in case of an old margin of the defective portion, it is desirable that the margin be freshened to promote tissue regeneration. Methods of freshening include, for example, a method wherein the epithelium of the margin is removed by bruising the margin using a surgical knife or the like, or by a treatment with a drug having a protein-denaturing effect, such as aluminum acetate or a high concentration of a local anesthetic.

Any suitable gelatin can be used in the gelatin sponge, including but not limited to crude collagen obtained by treating a bone, ligament, tendon, or skin of a bovine, pig, chicken, salmon or the like with an acid or alkali, thermally extracted with water, and the like, or mixtures thereof. The gelatin sponge can be treated in any way suitable for the intended purpose. For example, the gelatin may be cross-linked (using any suitable technique) to increase its water resistance. The gelatin sponge may contain another bioabsorbable polymeric material, as far as the function thereof is not adversely influenced. Such bioabsorbable polymeric materials include, but are not limited to, for example, synthetic polymers such as polylactic acid, polyglycolic acid, poly-ε-caprolactone, lactic acid-glycolic acid copolymer, glycolic acid-ε-caprolactone copolymer, lactic acid-ε-caprolactone copolymer, polycitric acid, polymalic acid, poly-acyanoacrylate, poly-β-hydroxy acid, polytrimethylene oxalate, polytetramethylene oxalate, polyortho-esters, poly-ortho-carbonates, polyethylene carbonate, poly-γ-benzyl-L-glutamate, poly-γ-methyl-L-glutamate, and poly-L-alanine; natural polymers such as polysaccharides such as starch, alginic acid, hyaluronic acid, chitin, pectic acid and derivatives thereof, and proteins such as gelatin, collagen, albumin, and fibrin, and the like. The gelatin sponge can be produced using any suitable technique, for example, by stirring and foaming an aqueous solution of gelatin using a homogenizer at a rotation rate of about 3000 to about 10000 rpm for about 10 seconds to about 5 minutes, then casting the aqueous solution of gelatin into a mold of an appropriate size, and 15 frozen at about −40 to about −80° C. for about 30 to about 120 minutes, thereafter freeze-drying this frozen matter under conditions of, for example, about 0.1 Torr. If the concentration of the aqueous solution of gelatin is too high, the softness of the gelatin sponge obtained decreases, so that it is preferable that the concentration be adjusted to, for example, about 3 w/w % or less. If further crosslinking is necessary, crosslinking can be performed as appropriate. Suitable gelatin sponges can be obtained from any source, including but not limited to Pfizer (GELFOAM®, comprising purified porcine skin, Gelatin USP granules and water).

Any suitable shape and size of the delivery device, such as a gelatin sponge, that is sufficient to cover the defective portion of the ear canal, such as the defective portion of the subject's tympanic membrane, can be used. The non-basic FGF, such as FGF-1, can be added to the delivery device via any suitable means. In one non-limiting embodiment, the FGF-1 is prepared as a liquid formulation and applied to the gelatin sponge. In another embodiment, the gelatin sponge is added to the FGF-1 formulation.

Any suitable dosage of non-basic FGF, such as FGF-1, can be used. In a preferred embodiment, the non-basic FGF is prepared as a liquid formulation at a concentration of between about 0.25 μg/ml and about 500 μm/mL; in various preferred embodiments, between 30 about 0.5 μm/mL and about 400 μm/mL; 0.75 μm/mL and about 300 μm/mL; 1 μm/mL and 200 μm/mL; 2.5 μm/mL and about 100 μm/mL; 25 μm/mL and about 500 μm/mL; 25 μm/mL and about 400 μm/mL; 25 μm/mL and about 300 μm/mL; 25 μm/mL and 200 μm/mL; and 25 μm/mL and about 100 μm/mL. The resulting delivery device can be used immediately, or may be stored as appropriate, such as by freeze-drying or lyophilizing.

The non-basic FGF, such as FGF-1, may be administered once and monitored for efficacy, or may be repeated (once, twice, etc., using the same or a different dosage) as deemed appropriate by an attending physician. The gelatin sponge containing the non-basic FGF may be administered to the ear canal defect for a period of time, such as between 1 hr and 6 months. The gelatin sponge may be on the ear canal defect continuously or near continuously for the period of time with occasional displacement to monitor the regeneration of the tympanic membrane. For example, the gelatin sponge may be applied to the perforated tympanic membrane for at least about 1 hr, at least about 24 hrs, at least about 2 days, at least about 7 days, at least about 2 weeks, at least about 3 weeks, at least about 4 weeks, at least about 1.5 months, at least about 2 months, at least about 3 months, at least about 4 months, at least about 5 months, or at least about 6 months.

### 8. PHARMACEUTICAL COMPOSITIONS

The non-basic FGF is typically administered as part of a pharmaceutical composition, which may further comprise, for example, lyoprotectants, surfactants, bulking agents, tonicity adjusting agents, stabilizers, preservatives and/or buffers. The non-basic FGF may be a component in a pharmaceutical composition. The pharmaceutical composition may also contain a pharmaceutically acceptable carrier. The pharmaceutical compositions comprising the non-basic FGF are for use in, but not limited to, monitoring a disorder or injury, in preventing, treating, managing, or ameliorating of a disorder or injury or one or more symptoms thereof, and/or in research. In a specific embodiment, a composition comprises one or more non-basic FGF. In another embodiment, the pharmaceutical composition comprises one or more non-basic FGFs and one or more prophylactic or therapeutic agents other than non-basic FGF for treating the subject suffering from a disorder or injury. In a further embodiment, the prophylactic or therapeutic agents are known to be useful for, or have been, or are currently being used in the prevention, treatment, management, or amelioration of a disorder or injury, or one or more symptoms thereof. In accordance with these embodiments, the composition may further comprise of a carrier, diluent, or excipient.

The non-basic FGF can be incorporated into pharmaceutical compositions suitable for administration to a subject. Typically, the pharmaceutical composition comprises a non-basic FGF and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives, or buffers, which enhance the shelf life or effectiveness of the non-basic FGF.

Various delivery systems are known and can be used to administer one or more non-basic FGF or the combination of one or more non-basic FGF and a prophylactic agent or therapeutic agent useful for preventing, managing, treating, or ameliorating a disorder or injury or one or more symptoms thereof, e.g., encapsulation in liposomes, microparticles, microcapsules, recombinant cells capable of expressing non-basic FGF or non-basic FGF variants, receptor-mediated endocytosis (see, e.g., Wu and Wu, J. Biol. Chem. 262:4429-4432 (1987)), construction of a nucleic acid as part of a retroviral or other vector, etc.

In a specific embodiment, it may be desirable to administer the non-basic FGF locally to the area in need of treatment; this may be achieved by, for example, and not by way of limitation, local infusion, by injection, or by means of an implant, said implant being of a porous or non-porous material, including membranes and matrices, such as sialastic membranes, polymers, fibrous matrices (e.g., Tissuel®), or collagen matrices. In one embodiment, an effective amount of one or more non-basic FGF is administered locally to the affected area to a subject to prevent, treat, manage, and/or ameliorate a disorder or a symptom thereof.

In a specific embodiment, where the composition is a nucleic acid encoding an non-basic FGF, the nucleic acid can be administered in vivo to promote expression of its encoded non-basic FGF, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see, e.g., Joliot et al., 1991, Proc. Natl. Acad. Sci. USA 88:1864-1868). Alternatively, a nucleic acid can be introduced intracellularly and incorporated within host cell DNA for expression by homologous recombination.

In a specific embodiment, nucleic acid sequences comprising nucleotide sequences encoding a non-basic FGF are administered to treat, prevent, manage, or ameliorate a disorder or one or more symptoms thereof by way of gene therapy. Gene therapy refers to therapy performed by the administration to a subject of an expressed or expressible nucleic acid. In this embodiment of the invention, the nucleic acids produce their encoded FGF that mediates a prophylactic or therapeutic effect.

Any of the methods for gene therapy available in the art can be used according to the present invention. For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, Science 260:926-932 (1993); and Morgan and Anderson, 1993, Ann. Rev. Biochem. 62:191-217; May, 1993, TIBTECH 11(5):155-215. Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990). Various methods of gene therapy are disclosed in US20050042664 A1 which is incorporated herein by reference.

Generally, the ingredients of compositions are supplied either separately or mixed together in unit dosage form, for example, as a dry lyophilized powder or water free concentrate in a hermetically sealed container such as an ampoule or sachette indicating the quantity of active agent. Where the mode of administration is infusion, composition can be dispensed with an infusion bottle containing sterile pharmaceutical grade water or saline. Where the mode of administration is by injection, an ampoule of sterile water for injection or saline can be provided so that the ingredients may be mixed prior to administration.

In particular, the invention also provides that one or more of the non-basic FGFs, or pharmaceutical compositions, is packaged in a hermetically sealed container such as an ampoule or sachette indicating the quantity of non-basic FGF. In one embodiment, one or more of the non-basic FGFs, or pharmaceutical compositions is supplied as a dry sterilized lyophilized powder or water free concentrate in a hermetically sealed container and can be reconstituted (e.g., with water or saline) to the appropriate concentration for administration to a subject. In one embodiment, one or more of the non-basic FGFs or pharmaceutical compositions is supplied as a dry sterile lyophilized powder in a hermetically sealed container at a unit dosage of at least 5 mg, for example at least 10 mg, at least 15 mg, at least 25 mg, at least 35 mg, at least 45 mg, at least 50 mg, at least 75 mg, or at least 100 mg. The non-basic FGF or pharmaceutical compositions should be stored at between 2° C. and 8° C. in its original container and the non-basic FGFs, or pharmaceutical compositions should be administered within 1 week, for example within 5 days, within 72 hours, within 48 hours, within 24 hours, within 12 hours, within 6 hours, within 5 hours, within 3 hours, or within 1 hour after being reconstituted. In an alternative embodiment, one or more of the non-basic FGFs or pharmaceutical compositions is supplied in liquid form in a hermetically sealed container indicating the quantity and concentration of non-basic FGF. In a further embodiment, the liquid form of the administered composition is supplied in a hermetically sealed container at least 0.25 mg/mL, for example at least 0.5 mg/mL, at least 1 mg/mL, at least 2.5 mg/mL, at least 5 mg/mL, at least 8 mg/mL, at least 10 mg/mL, at least 15 mg/mL, at least 25 mg/mL, at least 50 mg/mL, at least 75 mg/mL or at least 100 mg/mL. The liquid form should be stored at between 2° C. and 8° C. in its original container.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e., a non-basic FGF) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile, lyophilized powders for the preparation of sterile injectable solutions, methods of preparation comprise vacuum drying and spray-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including, in the composition, an agent that delays absorption, for example, monostearate salts and gelatin.

The pharmaceutical compositions may include a “therapeutically effective amount” or a “prophylactically effective amount” of a non-basic FGF. A “therapeutically effective amount” refers to an amount effective, at dosages and for periods of time necessary, to achieve the desired therapeutic result. A therapeutically effective amount of non-basic FGF may be determined by a person skilled in the art and may vary according to factors such as the disease state, age, sex, and weight of the individual, and the ability of non-basic FGF to elicit a desired response in the individual. A therapeutically effective amount is also one in which toxic or detrimental effects, if any, of non-basic FGF are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Dosage regimens may be adjusted to provide the optimum desired response (e.g., a therapeutic or prophylactic response). For example, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

An exemplary, non-limiting range for a therapeutically or prophylactically effective amount of non-basic FGF is a dose of between 0.1 and 200 mg/kg, for example between 0.1 and 10 mg/kg. The therapeutically or prophylactically effective amount of non-basic FGF may be between 1 and 200 mg/kg, 10 and 200 mg/kg, 20 and 200 mg/kg, 50 and 200 mg/kg, 75 and 200 mg/kg, 100 and 200 mg/kg, 150 and 200 mg/kg, 50 and 100 mg/kg, 5 and 10 mg/kg, or 1 and 10 mg/kg. It is to be noted that dosage values may vary with the type and severity of the condition to be alleviated. Further, the non-basic FGF dose may be determined by a person skilled in the art and may vary according to factors such as the disease state, age, sex, and weight of the individual, and the ability of the non-basic FGF to elicit a desired response in the individual. The dose is also one in which toxic or detrimental effects, if any, of the non-basic FGF are outweighed by the therapeutically beneficial effects. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The present invention has multiple aspects, illustrated by the following non-limiting examples.

### 9. EXAMPLES

The foregoing may be better understood by reference to the following examples, which are presented for purposes of illustration and are not intended to limit the scope of the invention.

### Example 1

Topical application of fibroblast growth factor-1 to the TMP of the study will result in closure of chronic TM perforations. Primary aims are: 1) to evaluate the safety and tolerability of FGF-1 to treat chronic non-healing tympanic membrane perforations; 2) to determine the maximum tolerated- or optimal biologic-dose of FGF-1 required to achieve complete closure of chronic non-healing tympanic membrane perforations; 3) to determine efficacy of TMP closure at the optimal biologic dose in a placebo controlled blinded phase II study.

Secondary aims are: 1) to determine the time to closure of tympanic membrane perforation as documented by otoscopy and by blinded photographic and tympanogram documentation; and 2) to measure blinded changes in pure-tone and speech discrimination scores pre and post-treatment. Patients will have complete recovery of tympanic membrane function without surgical intervention and reduction of recovery time.

Animal toxicology studies were completed. The findings in the animal study indicated high dose FGF-1 (7 μg) was well tolerated as assessed by clinical observations, body weight, auditory brainstem response, functional observation battery testing, hematology, clinical chemistry and ear histopathology. There did not appear to be any evidence of local toxicity or local irritation in the ear as a direct result of FGF-1 treatment indicating that FGF-1 treatment did not result in local or systemic toxicity or injury to the ear structures or to the hair cells under the conditions used in the study.

### Example 2

A gel foam section or gelatin sponge which has been saturated with FGF-1 alone or in combination with heparin will be administered by direct topical application to the tympanic membrane perforation. FGF-1 for administration to the tympanic membrane is formulated at the time of manufacture in sterile phosphate buffered saline with the addition of USP-grade heparin at a weight ratio of 3 parts heparin to 1 part FGF-1. The buffered FGF-1/Heparin solution is provided as a vialed, sterile parenteral solution. The vialed drug product is one component of a single patient, single dose clinical kit. 3 doses of FGF-1 will be tested.

In addition, there will be a placebo control group. The corresponding formulations of FGF-1 and the control are:

Control: Sterile phosphate-buffered saline (PBS)

Low dose: 25 μg FGF/1 mL of sterile PBS

Medium dose: 50 μg FGF/1 mL of sterile PBS

High dose: 100 μg FGF/1 mL of sterile PBS

Each formulation may also contain 150 mM sodium chloride, 10 mM sodium phosphate, pH 7.2 and a 3-fold excess of heparin to FGF-1 on a weight basis.

To anesthetize the tympanic membrane, a piece of gelfoam containing 1% lidocaine plus 1:1000 epinephrine will be placed on the perforated tympanic membrane for 5 minutes. The gelfoam will be removed and the edges of the perforation will be roughened using a Crabtree (right angle).

Administer FGF as follows: Moisten a small piece of gelfoam, the size of the perforation, with 0.2 mL of: 1.) Sterile phosphate-buffered saline (PBS) for the control group; 2.) Low does group: FGF solution (25 μg FGF-1 per mL of sterile PBS); 3.) High dose group: FGF solution (100 μg FGF-1 per 1 mL PBS). Insert over the perforation. The repair will be photographed. The subject will be advised to keep water out of the treated ear until the subject is seen again in follow-up.

The repair will be examined monthly. At the four week follow-up appointment the ear will be examined and photographed in the same fashion and the tympanic membrane will be examined to see if the perforation has closed.

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

What is claimed is:

1. A method for treating an ear canal tissue defect in a subject, the method comprising administering to the subject an amount effective of non-basic fibroblast growth factor (FGF) to treat the ear canal tissue defect.

2. The method of claim 1, wherein the non-basic FGF comprises FGF-1 (SEQ ID NO: 1) or a fragment thereof.

3. The method of claim 2, wherein the non-basic FGF comprises a polypeptide sequence of amino acids 15-155 of SEQ ID NO: 1.

4. The method of claim 2, wherein the non-basic FGF consists of a polypeptide sequence of amino acids 15-155 of SEQ ID NO: 1.

5. The method of claim 1, wherein the ear canal tissue defect comprises a perforated tympanic membrane.

6. The method of claim 2, wherein the non-basic FGF is administered to the perforated tympanic membrane.

7. The method of claim 6, wherein the non-basic FGF is administered using a delivery device.

8. The method of claim 7, wherein the delivery device is a gelatin sponge.

9. The method of claim 7, wherein the delivery device further comprises heparin.

10. The method of claim 7, wherein the delivery device comprises a covering material.

11. The method of claim 10, wherein the covering material comprises a fibrin glue.

12. The method of claim 1, wherein effective amount of non-basic FGF comprises between about 25 μg/mL and 100 μg/mL.

13. The method of claim 1, further comprising creating a fresh wound at the site of defect before administering the non-basic FGF.

14. A gelatin sponge coated or impregnated with a non-basic FGF.

15. The gelatin sponge of claim 14, wherein the non-basic FGF comprises FGF-1 (SEQ ID NO: 1) or a fragment thereof.

16. The gelatin sponge of claim 15, wherein the non-basic FGF consists of a polypeptide sequence of amino acids 15-155 of SEQ ID NO: 1.

17. The gelatin sponge of claim 14, wherein the gelatin sponge is impregnated with between about 25 μg/mL and about 100 μg/mL non-basic FGF.

18. The gelatin sponge of claim 14, wherein the gelatin sponge further comprises heparin.

19. The gelatin sponge of claim 14, wherein the gelatin sponge comprises a covering material.

20. The gelatin sponge of claim 19, wherein the covering material comprises a fibrin glue.
